(19) United States
(12) Patent Application Publication
(10) Pub. No.: US 2024/0115795 A1
(43) Pub. Date: Apr. 11, 2024

(54) VACUUM THERAPY DEVICES, KITS AND METHODS

(71) Applicants: Cambridge University Hospitals NHS Foundation, Cambridge (GB); Medovate Limited, Girton (GB)

(72) Inventors: Andrew Hindmarsh, Cambridge (GB); Alan Finnerty, Girton (GB); Mark Heath, Girton (GB); Robert Donald, Girton (GB); Stuart Thomson, Girton (GB)

(73) Assignees: Cambridge University Hospitals NHS Foundation, Cambridge, EN (GB); Medovate Limited, Girton, EN (GB)

(21) Appl. No.: 18/262,416

(22) PCT Filed: Jan. 24, 2022
(86) PCT No.: PCT/GB2022/050182

§ 371 Date: Jul. 21, 2023

(30) Foreign Application Priority Data
Jan. 22, 2021 (GB) 2100877.6

(51) Int. Cl. A61M 1/00; A61M 25/00

(52) U.S. Cl. CPC A61M 1/916; A61M 25/002

(57) ABSTRACT

A vacuum therapy device for treatment of a defect internal of a human or animal body, the device comprising: a suction tube extending from: (i) a proximal portion for coupling to a source of negative pressure outside the body, to (ii) a distal portion for providing suction proximal to the defect when inserted into the body; a porous medium coupled to the distal portion of the suction tube to provide suction to the defect when inserted into the body for treatment thereof; and a constraining sleeve for selective compression of the porous medium, the constraining sleeve having a proximal end and a distal end, wherein the constraining sleeve is movable relative to the porous medium between: an insertion position in which the proximal and distal ends of the sleeve are arranged at the distal portion of the suction tube so that the sleeve at least partially surrounds the porous medium to retain the porous medium in a compressed state during insertion into the body; and a treatment position in which the porous medium is in an expanded state for treatment of the defect.

### TECHNICAL FIELD

The present disclosure relates to devices, kits and methods for treatment of defects internal of a human or animal body, such as abscesses and abscess cavities. The present disclosure provides apparatuses, kits and methods for treating such internal defects through the application of a negative pressure at the site of the defect, e.g. to assist closure of an abscess cavity and/or to remove bodily fluids that may have accumulated at the defect. The present disclosure also provides apparatuses, methods and kits for preparing a device for vacuum therapy.

### BACKGROUND

Abscess cavities may include breaches in the continuity of the wall of the upper and lower gastrointestinal (GI) tract, which can create internal defects known as ‘leak cavities’. Such breaches may be a result of anastomotic leak or spontaneous/iatrogenic perforation, which can often result in severe sepsis. Traditionally, open surgery and/or radiological drainage is required to treat such defects, though this approach is often associated with high rates of morbidity and mortality, and furthermore may not always be feasible. It is estimated that around 50% of patients who have a leak from the upper gastrointestinal (GI) tract that requires surgical intervention do not recover.

Abscesses occurring in the peritoneal and pleural cavities usually occur due to bacterial infection within that cavity, for example following visceral perforation in the peritoneal cavity, such as perforated appendicitis or perforated diverticulitis, or following pneumonia or other insult such as penetrating trauma in the pleural cavity. It is recognised that drainage of the cavity (i.e. removing contaminants) can help to control infection at these internal defects, though drainage by way of surgery is associated with increased morbidity and mortality.

It is desirable to provide an apparatus and method for treating such internal defects that may avoid the need for open surgery.

WO 2017/182827 A1 discloses devices and methods for treatment of internal defects of a human or animal body. It discloses a catheter including a tube, an applicator and a porous medium, wherein the applicator can be controlled at a proximal end of the tube to deploy the porous medium from a distal end of the tube to treat the defect.

### SUMMARY

Aspects of the disclosure are set out in the independent claims and optional features are set out in the dependent claims. Aspects of the disclosure may be provided in conjunction with each other, and features of one aspect may be applied to other aspects.

In an aspect, there is provided a vacuum therapy device for treatment of a defect internal of a human or animal body. The device comprising: a suction tube extending from: (i) a proximal portion for coupling to a source of negative pressure outside the body, to (ii) a distal portion for providing suction proximal to the defect when inserted into the body; a porous medium coupled to the distal portion of the suction tube to provide suction to the defect when inserted into the body for treatment thereof; and a constraining sleeve for selective compression of the porous medium, the constraining sleeve having a proximal end and a distal end. The constraining sleeve is movable relative to the porous medium between: an insertion position in which the proximal and distal ends of the sleeve are arranged at the distal portion of the suction tube so that the sleeve at least partially surrounds the porous medium to retain the porous medium in a compressed state during insertion into the body; and a treatment position in which the porous medium is in an expanded state for treatment of the defect.

Embodiments may provide a device which has a distal portion arranged to facilitate easier insertion into a human or animal body. The sleeve may retain the porous medium in a compressed state so that it takes up less volume and causes fewer difficulties when being passed into the body (e.g. through a patient's nose). The sleeve may be removable from the porous medium to enable the porous medium to then return to its expanded state for treatment of the defect. For example, the sleeve may be configured to slide over the porous medium (distally or proximally), or to be ripped (e.g. torn) away from the porous medium. The device may be configured to facilitate easy removal of the sleeve, such as after the distal end of the device has passed through a subject's nasal passageway.

The constraining sleeve may be configured to be inserted through a nasal passageway of a human. The constraining sleeve may be configured to retain the porous medium in its compressed state during insertion of said porous medium and the distal portion of the suction tube through the nasal passageway. The device may be arranged to enable expansion of the porous medium to its expanded state once the porous medium and distal portion of the suction tube have passed through the nasal passageway. The device may be configured to enable the distal portion of the suction tube, the porous medium and the constraining sleeve to be passed out through the human mouth after being inserted through their nasal passageway for movement of the constraining sleeve from the insertion position to the treatment position.

The constraining sleeve may comprise a gripping region and/or a gripping element configured to be gripped for movement of the constraining sleeve from the insertion position to the treatment position. The gripping element of the constraining sleeve may comprise a tab. The tab may extend radially outward from the constraining sleeve. The porous medium, the constraining sleeve and the distal portion of the suction tube may be configured to be inserted into a tube of a nasal insertion applicator and/or endoscope for placement into the body. The constraining sleeve may be affixed to the suction tube, e.g. to hold it in place at the distal portion of the suction tube. For example, the constraining sleeve may be arranged to retain the porous medium in its compressed state during insertion into the body (e.g. when passing through a nasal insertion applicator, or when passing into the body directly).

The constraining sleeve may comprise means for removal through the suction tube. For example, the constraining sleeve may be arranged to enable it to be pulled through the suction tube. The constraining sleeve may be removed while inside a subject's body, and retracted from the body (e.g. by pulling the sleeve through the suction tube). For example, the sleeve may have a connection region for coupling to a component for pulling the sleeve through the suction tube, e.g. the component may also be configured for removal of the sleeve. For example, the connection region may comprise a region configured to be gripped or a tab, e.g. which is configured to be grasped. The constraining sleeve may be arranged so that the connection region may be grasped to enable the constraining sleeve to be removed from the porous medium and/or to pull the constraining sleeve into the suction tube (e.g. for removal through the proximal end of the suction tube. For example, removal of the constraining sleeve may comprise removing the sleeve inside the body and retracting the sleeve through the suction tube to outside the body.

The device may comprise a nasal insertion applicator having an internal lumen which provides the constraining sleeve. The nasal insertion applicator may be configured to be inserted into a human nasal passageway to enable movement of the porous medium and the distal portion of the suction tube through the inner lumen and into the body with the porous medium in its compressed state. The constraining sleeve may comprise a nasal insertion applicator configured to provide a tube through a human nasal passageway to enable movement of the porous medium and the distal portion of the suction tube through the nasal passageway with the porous medium in its compressed state.

The constraining sleeve may comprise an inner lining configured to reduce friction between the sleeve and the porous medium. The constraining sleeve may be small relative to the length of the suction tube. The constraining sleeve may be less than half the length of the suction tube. The constraining sleeve may be configured to move distally relative to the suction tube from the insertion position to the treatment position or to be torn away from the porous medium. The device may be arranged so that movement of the constraining sleeve from the insertion position to the treatment position causes the porous medium to expand into its expanded state for treatment of the defect. The suction tube may comprise a vacuum coupling for connection to a pump to provide suction to the porous medium. The suction tube may be a single tube which extends from the vacuum coupling to the porous medium. The constraining sleeve may be configured to shrink in response to suction being provided within the sleeve. The constraining sleeve may be configured to shrink and compress the porous medium into its compressed state when the sleeve surrounds the porous medium and suction is applied to the porous medium by the suction tube.

In an aspect, there is provided a kit of parts for a vacuum therapy device for treatment of a defect internal of a human or animal body. The kit comprising: a suction tube extending from: (i) a proximal portion for coupling to a source of negative pressure outside the body, to (ii) a distal portion for providing suction proximal to the defect when inserted into the body; a porous medium couplable to the distal portion of the suction tube to provide suction to the defect when the device is assembled and inserted into the body for treatment thereof; and a constraining sleeve configured to be: (i) placeable into an insertion position in which proximal and distal ends of the sleeve are arranged at the distal portion of the suction tube with the sleeve at least partially surrounding the porous medium to retain the porous medium in a compressed state during insertion into the body, and (ii) movable from the insertion position to a treatment position in which the porous medium is in an expanded state for treatment of the defect.

The kit may comprise a funnel having an inlet providing an inlet cross-sectional area and an outlet providing an outlet cross-sectional area. The outlet cross-sectional area may be less than the inlet cross-sectional area. The outlet cross-sectional area may be selected so that movement of the porous element through the funnel from the inlet to the outlet compresses the porous medium to a size at which it fits into the constraining sleeve. The funnel may comprise a channel in its surface from the inlet to the outlet. The channel may be arranged to enable the funnel to be removed from the suction tube after the porous medium has been compressed and inserted into the constraining sleeve. The kit may comprise an insertion applicator for insertion into a subject's nasal passageway. The constraining sleeve may be configured for movement within a tube of the insertion applicator. The tube may comprise a friction-reducing coating on its inner surface.

In an aspect, there is provided a kit of parts for nasal insertion of a vacuum therapy device into a human or animal body for treatment of a defect in the body. The kit comprising: a suction tube extending from: (i) a proximal portion for coupling to a source of negative pressure outside the body, to (ii) a distal portion for providing suction proximal to the defect when inserted into the body; a porous medium couplable to the distal portion of the suction tube to provide suction to the defect when the device is assembled and inserted into the body for treatment thereof; and a nasal insertion applicator having an internal lumen which provides a constraining sleeve for selective compression of the porous medium, wherein the nasal insertion applicator is configured to be placeable into an insertion position within a nasal passageway of the body to enable the distal portion of the suction tube and the porous medium to pass through the applicator into the body while retaining the porous medium in a compressed state during insertion into the body.

In an aspect, there is provided a method of preparing a vacuum therapy device for treatment of a defect internal of a human or animal body, the device comprising: (i) a suction tube extending from a proximal portion for coupling to a source of negative pressure outside the body to a distal portion for providing suction proximal to the defect when inserted into the body, and (ii) a porous medium coupled to the distal portion of the suction tube to provide suction to the defect when inserted into the body for treatment thereof, the method comprising: providing the porous medium in a compressed state within a constraining sleeve with the sleeve in an insertion position in which proximal and distal ends of the sleeve are arranged at the distal portion of the suction tube so that the sleeve at least partially surrounds the porous medium to enable the sleeve to retain the porous medium in the compressed state during insertion into the body, wherein the constraining sleeve is movable relative to the porous medium between the insertion position and a treatment position in which the porous medium is in an expanded state for treatment of the defect.

Providing the porous medium in the compressed state within the constraining sleeve may comprise placing the constraining sleeve over the porous medium and applying suction to the porous medium through the suction tube to shrink the sleeve. Providing the porous medium in the compressed state within the constraining sleeve may comprise moving the porous medium through a funnel from a larger cross-sectional area to a smaller cross-sectional area, wherein at the smaller cross-sectional area the porous medium is in its compressed state and it fits within the constraining sleeve.

In an aspect, there is provided a vacuum therapy device for treatment of a defect internal of a human or animal body. The device has a distal portion for insertion into the body for treating the defect. The device comprises: a porous medium for treatment of the defect, wherein the porous medium is located at the distal portion of the device; a suction tube extending coupled to the porous medium at the distal portion of the device to provide suction thereat; and a distal constraining tip having a proximal end arranged at the distal portion of the device, the distal constraining tip comprising a constraining sleeve configured to retain the porous medium in a compressed state for insertion into the body. The constraining sleeve is movable between: (i) an insertion position in which the sleeve at least partially surrounds the porous medium and retains the porous medium in the compressed state, and (ii) a treatment position in which the porous medium is in an expanded state for treatment of the defect.

The constraining sleeve may comprise a gripping region and/or a gripping element configured to be gripped for movement of the constraining sleeve from the insertion position to the treatment position. The gripping element of the constraining sleeve comprises a tab, such as a tab which extends radially outward from the constraining sleeve. Gripping regions and/or elements may be configured to provide a pre-defined region or element to facilitate with removal of the sleeve from the porous medium. For example, gripping elements may comprise a portion of the sleeve which is arranged to be pulled/pushed by a user for sliding the sleeve over an outer surface of the porous medium. For example, a gripping region may comprise a region of the sleeve connected to, or adjacent to, a pre-defined tearing region for the sleeve, e.g. to provide a portion of the sleeve to be pulled/pushed to cause the pre-defined tearing region to tear, thereby to facilitate removal (by tearing) of the sleeve from the porous medium.

The distal portion of the device may be configured to be inserted through a body lumen of the human nose, such as through the nasal cavity. For example, the device may be sized and shaped to fit within the nasal passageway, and/or the device may be flexible enough to bend round passages of the nasal passageway towards the throat. The sleeve may be sized and shaped to fit within the nasal passageway, and resistive enough to expansion of the porous medium (e.g. radial expansion) to inhibit expansion of the sleeve to a size and/or shape which would not fit within the nasal passageway. The distal portion of the device may be configured to be inserted into a human nose and passed back out through their mouth to enable the constraining sleeve to be removed from the porous medium prior to further advancing the porous medium and suction tube into the body towards the defect. The porous medium, distal constraining tip and suction tube may be configured to be inserted into a tube of an insertion applicator for placement into the body. The tube of the insertion applicator may comprise a tube running through a patient's nose or an endoscope. The suction tube may be stiffer than the porous medium. The Young's modulus of the suction tube may be greater than that of the porous medium.

The distal constraining tip may be small relative to the length of the suction tube. The distal constraining tip may extend along less than half of the length of the suction tube. For example, the distal constraining tip may be less than 100 mm, e.g. less than 50 mm, e.g. less than 40 mm, e.g. less than 30 mm, e.g. less than 20 mm. The constraining sleeve may be configured to move distally relative to the suction tube from the insertion position to the treatment position or to be torn away from the porous medium. The device may be arranged so that movement of the constraining sleeve from the insertion position to the treatment position causes the porous medium to expand into its expanded state for treatment of the defect. The constraining sleeve may comprise at least one of: (i) a material configured to not degrade or dissolve during use, and (ii) a mesh or a resilient material, such as rubber. The suction tube may comprise a vacuum coupling for connection to a pump to provide suction to the porous medium. The suction tube may be a single tube which extends from the vacuum coupling to the porous medium.

The constraining sleeve may be configured to shrink in response to suction being provided within the sleeve. The constraining sleeve is configured to shrink and compress the porous medium into its compressed state when the sleeve is in the insertion position and suction is applied to the porous medium by the suction tube. For example, the device may be arranged so that, once the sleeve has been shrunk, it retains a sufficiently airtight seal to retain the porous medium in its compressed state. The device may comprise one or more graspable elements, such as sutures, located at the proximal end and/or a distal end of the porous medium.

A hollow passageway may extend through at least a portion of the porous medium. A portion of the suction tube may extend at least partially within the hollow passageway of the porous medium. The suction tube may comprise a plurality of fenestrations along the length of the portion within the hollow passageway of the porous medium to provide suction at a plurality of different locations along the porous medium. Fenestrations may enable a more uniform application of negative pressure at the porous medium. This may provide a more uniform application of suction to the defect, and more uniform distribution of healing of tissue at the defect. The fenestrations may enable suction to be provided to the porous medium even if other fenestrations have been blocked (e.g. by matter inside the body). The plurality of fenestrations may extend along the portion of the suction tube within the hollow passageway of the porous medium. The hollow passageway may comprise a hollow core extending along a central axis of the porous medium (e.g. along a longitudinal axis of the porous medium, such as within the middle of the porous medium). The porous medium may be deformable to fit within the constraining sleeve. The porous medium may be configured so that at least a portion of the porous medium retains its pre-deformed shape when removed from the constraining sleeve.

The fenestrations may have rounded corners. For example, the fenestrations may be polygonal with rounded corners, such as square shaped with rounded corners, or the fenestrations may be more rounded, such as ovaloid or circular in shape. Rounded corners of the fenestrations may reduce the likelihood of tearing of the suction tube. The fenestrations may be arranged in the suction tube in a stepped manner, so that adjacent fenestrations are both laterally and longitudinally offset from each other, e.g. the fenestrations may be arranged in different regions along the length of the suction tube, as well as in different regions around the circumference of the suction tube. The suction tube may have five or more fenestrations, such as ten or more, such as 15 or more. For example, each fenestration may have a greater area than the area of the hole at the end of the suction tube. The fenestrations may have a total open area of at least 10 mm², such as at least 10 mm², such as at least 10 mm², such as at least 20 mm², such as at least 25 mm², such as at least 30 mm², such as at least 35 mm², such as at least 40 mm². For example, each individual fenestration may have an open area of at least 0.5 mm², such as at least 1 mm², such as at least 1.25 mm², such as at least 1.5 mm², such as at least 1.75 mm², such as at least 2 mm².

The device may be an endoluminal vacuum therapy device configured for treatment of an abscess in the gastrointestinal tract. The device may be configured for insertion into the gastrointestinal tract through a nose or mouth of the human or animal body. The defect may be an abscess cavity, possibly caused by a breach in the wall of the lower or upper gastrointestinal (Cl) tract, including in the pharynx and oesophagus, whereby the device may be adapted for insertion into the body to access the defect endoluminally. The device may comprise a pump (e.g. a vacuum apparatus) connected to the suction tube (whether directly or indirectly). The device may be configured so that the suction tube may be connected with the pump to provide suction (e.g. negative pressure) at the porous medium. The device may comprise a receptacle coupled to the suction tube. The receptacle may be configured to receive substances sucked up through the suction tube. The suction tube may be arranged for connection to one or more fluid delivery devices. For example, the suction tube may be arranged to be connected to a syringe for providing fluids to the device, such as a lubricant for facilitating relative movement between the suction tube and e.g. a tube surrounding the suction tube such as for insertion into a patient's nose, or a fluid for flushing tissue from the porous medium (e.g. saline). Suction tubes of the present disclosure may be formed from silicon, or a suitable polymer such as PVC (polyvinyl chloride).

The suction tube may comprise a suction component at least partially within the hollow passageway of the porous medium. The suction component may comprise one or more open channels extending along a majority of the length of the porous medium to provide suction at along the length of the porous medium. For example, the suction component may be cruciate shaped in cross section. The cruciate shape may define a plurality of channels extending along a length of the component. For example, the cruciate shape may comprise a plurality of flanges extending radially outward, such as extending radially outward from a central region of the suction component. Each of the radially extending flanges may also comprise a lip at their radially outer end, wherein the lip extends circumferentially from the radially outer end of the flange. The spacing between adjacent flanges and their respective lips may define the channels. For example, because each flange and its respective lip(s) do not touch adjacent flanges/lips, the suction component will define channels which are only partially sealed, e.g. so that suction applied to the suction component at its proximal end (e.g. by the inner tube) may be transferred to each channel at the proximal end. Due to the channels not being sealed at each radial point along their length, suction will be transferred out through the channels to porous medium adjacent the channels. The suction component is shaped so that the suction is delivered along its length (to surrounding porous medium). For example, the suction component may be shaped like a cross potent (e.g. crutch cross) when viewed in cross section. The cross bars at the four ends of the cross may be curved (as if sections of a circumference of a circle connecting the four ends). The four ends may extend toward each other, but not so far as to touch each other.

In an aspect, there is provided a kit of parts for a vacuum therapy device for treatment of a defect internal of a human or animal body. When assembled the vacuum therapy device has a distal portion for insertion into the body for treating the defect. The kit comprises: a porous medium for treatment of the defect; a suction tube couplable to the porous medium to provide suction thereat, wherein the suction tube is couplable to the porous medium with the porous medium located at the distal portion of the device; and a distal constraining tip comprising a constraining sleeve, wherein the constraining sleeve is configured to be: (i) placeable over the porous medium into an insertion position in which a proximal end of the sleeve is arranged at the distal portion of the device with the sleeve at least partially surrounding the porous medium and retaining the porous medium in a compressed state for insertion into the body, and (ii) slidable over an outer surface of the porous medium to move from the insertion position into a treatment position in which the porous medium is in an expanded state for treatment of the defect. For example, when assembled the kit may provide a vacuum therapy device of the present disclosure.

The kit may comprise a compression element operable to compress the porous medium into its compressed state. For example, the compression element may comprise a separate component for facilitating compression of the porous medium, or the compression element may include a mechanism for using one or more components of the device to compress the porous medium. The compression element may comprise a funnel having an inlet providing an inlet cross-sectional area and an outlet providing an outlet cross-sectional area, wherein the outlet cross-sectional area may be less than the inlet cross-sectional area. The outlet cross-sectional area may be selected so that movement of the porous element through the funnel from the inlet to the outlet compresses the porous medium to a size at which it fits into the constraining sleeve. The funnel may comprise a channel in its surface from the inlet to the outlet. The channel may be arranged to enable the funnel to be removed from the suction tube after the porous medium has been compressed and inserted into the constraining sleeve. The sleeve may be suitably rigid to provide the compression element (e.g. by forcing the porous medium into the sleeve the porous medium may be compressed). Operation of the pump to apply suction to the porous medium may provide the compression element by collapsing the sleeve onto the porous medium to compress it.

The kit may comprise an insertion applicator for insertion into a subject's nasal passageway. The constraining sleeve may be configured for movement within a tube of the insertion applicator. The insertion applicator may be arranged to inhibit friction between its inner surface and the outer surface of the device, e.g. the outer surface of the suction tube and sleeve (with porous medium inside). The insertion applicator may comprise a friction-reducing coating on its inner surface. For example, the insertion applicator may comprise an inner lining arranged to inhibit friction between the suction tube/sleeve and the insertion applicator. The inner lining may be made of a material arranged to reduce friction, such as fluorinated ethylene propylene. The inner lining may be adhered, or sewed, to the inside of the catheter, and/or it may just sit within the catheter without being affixed thereto. The suction tube and/or sleeve may have a coating on its outside surface configured to reduce friction with the inner surface of the insertion applicator.

In an aspect, there is provided a method of preparing a vacuum therapy device for treatment of a defect internal of a human or animal body. The device has a distal portion for insertion into the body for treating the defect. The device comprises: (i) a porous medium located at a distal portion of the device for treatment of the defect, and (ii) a suction tube coupled to the porous medium at the distal portion of the device to provide suction thereat. The method comprises: compressing the porous medium into a compressed state; and arranging the porous medium within a distal constraining tip having a proximal end arranged at the distal portion of the device, wherein the distal constraining tip comprises a constraining sleeve configured to retain the porous medium in a compressed state for insertion into the body, wherein arranging the porous medium within the distal constraining tip comprises arranging the constraining sleeve in an insertion position in which it at least partially surrounds the porous medium and retains the porous medium in the compressed state for insertion into the body, wherein the constraining sleeve is movable between the insertion position and a treatment position in which the porous medium is in an expanded state for treatment of the defect.

Compressing the porous medium into the compressed state may comprise placing the constraining sleeve over the porous medium into the insertion position and applying suction to the porous medium through the suction tube to shrink the sleeve. Compressing the porous medium into the compressed state may comprise forcing the porous medium into the constraining sleeve so that it is compressed into its compressed state. Compressing the porous medium into the compressed state may comprise moving the porous medium through a funnel from a larger cross-sectional area to a smaller cross-sectional area, wherein at the smaller cross-sectional area the porous medium is in its compressed state and it fits within the constraining sleeve.

### FIGURES

Some examples of the present disclosure will now be described, by way of example only, with reference to the figures, in which:

FIGS. 1a and 1b show schematic diagrams of an exemplary vacuum therapy device.

FIGS. 2a and 2b show schematic diagrams illustrating an exemplary method for preparing a vacuum therapy device.

FIGS. 3a and 3b show schematic diagrams illustrating an exemplary method for preparing a vacuum therapy device.

FIGS. 4a, 4b, 4c and 4d show schematic diagrams illustrating an exemplary method for preparing a vacuum therapy device.

In the drawings like reference numerals are used to indicate like elements.

Specific Description

Embodiments are directed to vacuum therapy devices for treatment of a defect within a subject's body. A suction tube is provided with a porous medium at a distal portion of the suction tube. The device may be used to place the porous medium in, or near to, the defect, and a negative pressure is applied through the suction tube to the porous medium to facilitate healing of the defect. To facilitate insertion of the device into the body, a constraining tip may be provided which retains the porous medium in a compressed state. The compressed porous medium and suction tube may be inserted into a subject's body as part of the method for placing the porous medium at the defect. For example, the constraining sleeve may enable the porous medium to be retained in a sufficiently compressed state that the porous medium and suction tube may be inserted through a patient's nasal passageway. The constraining tip may subsequently be removed from the porous medium, e.g. either outside the patient's body (after having been passed out their mouth) or inside the patient's body (such as at or near to the defect). The constraining tip may be physically removed from the porous medium to enable the porous medium to then expand for treatment of the defect.

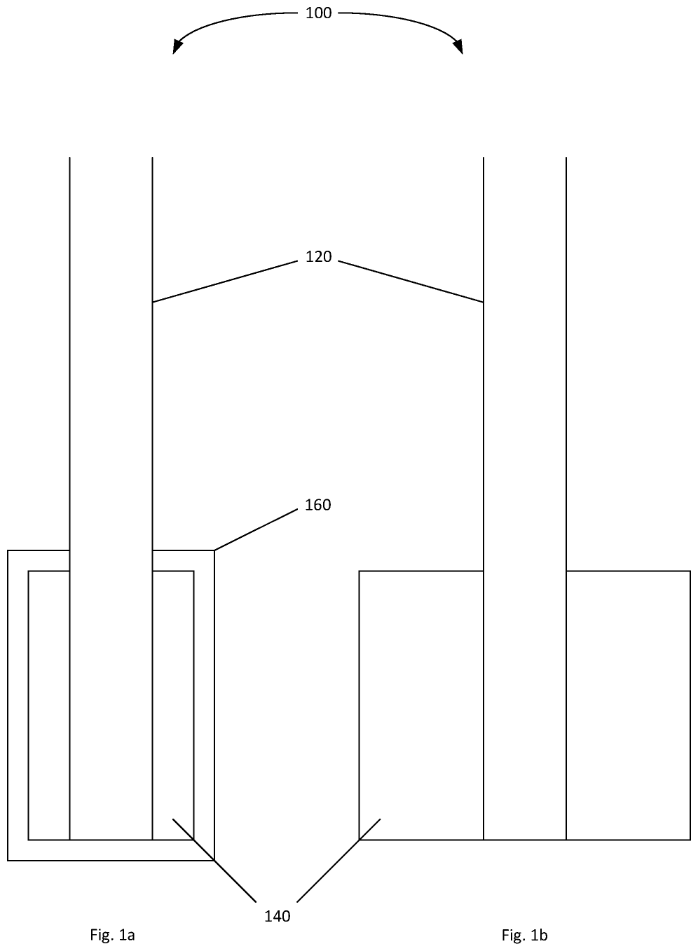

An exemplary vacuum therapy device will now be described with reference to FIGS. 1a and 1b.

FIGS. 1a and 1b show a vacuum therapy device 100. The device 100 includes a suction tube 120 and a porous medium 140. The device 100 also includes a distal constraining tip shown as a constraining sleeve 160.

FIG. 1a shows the device 100 with the constraining sleeve 160 on the porous medium 140, and the porous medium 140 in a constrained (compressed) state. FIG. 1b shows the device 100 with no constraining sleeve 160 on the porous medium 140, and the porous medium 140 in a non-constrained (expanded) state.

The porous medium 140 is located at a distal portion of the suction tube 120. In the example shown in FIGS. 1a and 1b, the porous medium 140 is arranged at a distal tip of the suction tube 120. The suction tube 120 extends from a proximal region (not shown) to a distal region. In use, the proximal portion will be outside the body and the distal portion will be inside the body. The suction tube 120 extends through a hollow passageway of the porous medium 140. A distal tip of the suction tube 120 is located proximal to a distal end of the porous medium 140. The porous medium 140 surrounds a portion of the length of the suction tube 120. The porous medium 140 extends from its distal end (proximal to the distal tip of the suction tube 120) to its proximal end. The proximal end of the porous medium 140 is in the distal region of the suction tube 120. The length of the porous medium 140 is less than that of the suction tube 120.

The constraining sleeve 160 of the distal constraining tip is located at a distal portion of the suction tube 120. The length of the distal constraining tip corresponds to the length of the porous medium 140. Typically, the length of the distal constraining tip will be more than the length of the porous medium 140, but less than that of the suction tube 120. The constraining sleeve 160 is arranged at the distal region of the suction tube 120. A proximal and distal region of the constraining sleeve 160 is arranged at the distal region of the suction tube 120. Both the porous medium 140 and the constraining sleeve 160 surround the suction tube 120. The constraining sleeve 160 surrounds the porous medium 140 which surrounds the suction tube 120. Both the constraining sleeve 160 and the porous medium 140 are arranged at the distal tip of the suction tube 120. Both the porous medium 140 and the constraining sleeve 160 are small in length relative to the suction tube 120, e.g. they only extend along a small portion of the length of the suction tube 120.

In FIG. 1a, the device 100 is shown with the porous medium 140 in a compressed state. Both the constraining sleeve 160 and the porous medium 140 are at the distal end of the suction tube 120, with the constraining sleeve 160 surrounding the porous medium 140. The constraining sleeve 160 is arranged in an insertion position in which it is aligned with the porous medium 140. In the insertion position, the constraining sleeve 160 surrounds the porous medium 140 along the majority of the length of the porous medium 140 (all of the length of the porous medium 140, as shown in FIGS. 1a and 1b). The constraining sleeve 160 surrounds the porous medium 140 around the perimeter of the porous medium 140. With the constraining sleeve 160 in the insertion position, the porous medium 140 is retained in a compressed state.

In FIG. 1b, the device 100 is shown with the porous medium 140 in its expanded state. There is no constraining sleeve 160 in FIG. 1b to retain the porous medium 140 in its compressed state. As such, the porous medium 140 has expanded in volume. The porous medium 140 has expanded radially so that the porous medium 140 is wider in FIG. 1b. The constraining sleeve 160 is not shown in FIG. 1b. As discussed in more detail below, the constraining sleeve 160 will have been moved relative to the porous medium 140. This may comprise sliding the sleeve 160 over the porous medium 140 (either proximally or distally) and/or tearing the sleeve 160 away (e.g. ripping it off the porous medium 140).

The vacuum therapy device 100 in this example is an endoscopic vacuum therapy (EVT) device. Endoscopic vacuum therapy is a relatively new technique for treating defects, such as oesophageal perforation and certain other leakages from the UGI tract, such as post-operative leakages. EVT is a minimally invasive, alternative method of treatment to traditional surgery, utilising vacuum-assisted closure (VAC) techniques. EVT involves placing a porous medium, such as a polyurethane sponge, into a defect cavity under endoscopic visualization and then applying a continuous negative pressure, causing the cavity to collapse around the sponge. The sponge is typically changed every 48-72 hours until the cavity shrinks and stable granulation tissue forms a barrier.

EVT includes three different stages for treating a gastrointestinal defect. For example, EVT may be used to treat a defect in the oesophagus. To treat the defect, a tube may be inserted through the nose or mouth and then directed to the defect under direct endoscopic visualisation. A porous medium may be carried into the patient using the tube and then placed in the defect cavity, or a lumen proximal thereto, such as a lumen of the bowel (e.g. for intra-luminal vacuum therapy). A negative pressure, such as −125 mm Hg, may then be applied, causing the defect cavity to collapse around the porous medium to aid healing. This treatment may also be referred to as endoscopic ‘transluminal’ or ‘intraluminal’ vacuum therapy.

The vacuum therapy device 100 is configured to be inserted into a human or animal body for treatment of the defect internal the body. The device 100 is configured to be inserted into the body with the porous medium 140 in, or proximal to, the defect. With the porous medium 140 inserted into the relevant region of the body, the device 100 will have a proximal region outside the body. The device 100 is arranged so that the porous medium 140 and the constraining sleeve 160 are in the distal portion of the device 100 so that they will be inside the body in use. For example, the constraining sleeve 160 may be arranged so that it does not extend outside the body when it surrounds the porous medium 140.

Although not shown in the figures, the suction tube 120 may be connected to a source of negative pressure at its proximal end. This connection to the source of negative pressure may be direct or indirect (via one or more additional components for transmitting negative pressure). The device 100 is configured to transmit such negative pressure, so that it may be applied at the porous medium 140 to provide suction thereat. For example, a proximal end of the inner suction 120 may be secured to an adaptor for connection to a source of negative pressure, such as a vacuum apparatus. The device 100 may be arranged so that when in use, such an adaptor is located outside of the patient's body. The adaptor may provide a connector for coupling the suction tube 120 with the vacuum apparatus. The adaptor may be provided with a further connector, which is substantially in-line with the other connector, for coupling with a flexible tube (e.g. formed from FEP). The further connector may be provided with barbs, which a flexible tube may be stretched over so as to provide a secure fluid-tight coupling. Any suitable type of connector may be used, such as a lock connector, e.g. a luer lock connector.

The suction tube 120 is configured to support the porous medium 140 at its distal end. The suction tube 120 is configured to transmit suction to (e.g. to provide negative pressure at) the porous medium 140. The suction tube 120 is configured to receive air or other substances which are transmitted into the suction tube 120 through the porous medium 140 and to transmit these away. The suction tube 120 is configured to be flexible with an atraumatic tip (e.g. to inhibit the suction tube 120 from causing damage to a patient when being moved around inside the patient). The suction tube 120 may extend into a hollow passageway of the porous medium 140. An outer surface of the suction tube 120 may be coated with an adhesive to facilitate a stronger connection between the suction tube 120 and the porous medium 140 (e.g. in the region of suction tube 120 which lies in the hollow passageway of the porous medium 140. A tip of the suction tube 120 may be coupled (e.g. via a wire or suture) to a component at a proximal location in the device 100 to improve control of the movement of the suction tube 120, such as to facilitate removal of the suction tube 120 and porous medium 140 from a defect.

The porous medium 140 is arranged to facilitate treatment of the defect internal of the human body by application of a negative pressure (e.g. providing suction) through the porous medium 140 at the defect. The porous medium 140 is configured so that a negative pressure applied to it from the suction tube 120 will be transmitted to the surface of the porous medium 140. The device 100 may be configured so that suction is applied to the environment surrounding the porous medium 140. The porous medium 140 may be configured to facilitate tissue ingrowth for tissue in and around the defect. Tissue in the environment of the porous medium 140 may be drawn towards the porous medium 140 (due to the suction), and the porous medium 140 is configured to be biocompatible for tissue coming into contact therewith.

The porous medium 140 has a hollow passageway in which the suction tube 120 is provided and connected thereto. The porous medium 140 is sized so that it is compressible to fit within the constraining sleeve 160, and e.g. for insertion into the patient such as via their nasal passageway. The porous medium 140 comprises a material having pores which are typically of a size between 400 to 600 microns. The porous medium 140 may include one or more materials such as: (i) foams e.g. a polyurethane foam, (ii) expandable meshes e.g. a wire mesh, (iii) bio-active materials e.g. bio-active collagen. For example, the wire mesh may be formed of a shape memory material, such as a nickel titanium alloy (e.g. nitinol). The porous medium 140 is configured to be compressed to a volume at which it is retainable in the constraining sleeve 160, and from which it will expand to its pre-compressed volume.

The constraining sleeve 160 is configured to retain the porous medium 140 in its compressed state when surrounding the porous medium 140 on the suction tube 120. The constraining sleeve 160 and the porous medium 140 are configured so that in the absence of the constraining sleeve 160 surrounding the porous medium 140, the porous medium 140 will expand to its expanded state. The constraining sleeve 160 is configured so that, when located in its insertion position, it retains the porous medium 140 in a compressed state. The constraining sleeve 160 is configured to press the porous medium 140 against the suction tube 120 so that it takes up less volume.

The constraining sleeve 160 and porous medium 140 are configured so that when the sleeve 160 is moved away from its insertion position, the porous medium 140 will expand. The constraining sleeve 160 is arranged to be movable relative to the porous medium 140 (e.g. to enable movement of the sleeve 160 from the insertion position to a different position such as a treatment position). Movement of the constraining sleeve 160 relative to the porous medium 140 may comprise mechanical (e.g. physical) movement. For example, the sleeve 160 may be configured to slide over an outer surface of the porous medium 140. Sliding may be in either a distal direction or a proximal direction relative to the porous medium 140. For example, the sleeve 160 may be configured to be tearable, e.g. to facilitate removal by tearing (ripping) the sleeve 160 off the porous medium 140. The sleeve 160 is arranged to facilitate user removal (whether by hand, or using another implement, such as graspers of an endoscope) from the insertion position on the porous medium 140. The sleeve 160 comprises a mesh and/or a resilient material configured not to degrade/dissolve during use (e.g. in response to saline or other solutions which may be present during use). For example, the sleeve 160 may be made of rubber or silicon (or other suitable plastic materials).

Two exemplary methods of using the device 100 of FIGS. 1a and 1b will now be described.

In a first exemplary method, the device 100 is arranged with the constraining sleeve 160 in the insertion position and the porous medium 140 in its compressed state on the suction tube 120. The distal portion of the device 100 (the porous medium 140, constraining sleeve 160 and the distal end of the suction tube 120) are inserted into the subject's nasal passageway (e.g. through a nostril). The distal portion of the device 100 is advanced towards the throat and back out through the subject's mouth. Once outside the subject's body, the constraining sleeve 160 is moved from its insertion position on the porous medium 140. Moving the constraining sleeve 160 from the insertion position may comprise pulling (e.g. tearing) the sleeve 160 away from the porous medium 140, or sliding the sleeve 160 away from the insertion position to a treatment position where it no longer surrounds the porous medium 140. For example, the sleeve 160 may be slid distally relative to the porous medium 140 such as to remove it from the device 100.

The porous medium 140 will then expand to its expanded state for treatment of the defect. For treatment, the distal end of the suction tube 120 will then be re-inserted into the subject's mouth with the constraining sleeve 160 removed and the porous medium 140 in its expanded state. The distal portion of the device 100 is then advance through the mouth and throat and into the gastrointestinal tract where it may be placed suitably for treatment of the defect. For example, the porous medium 140 may be located in, or proximal to, the defect. Once located in the body for treatment of the defect, suction is applied to the defect.

For this, a pump or vacuum source is coupled to the suction tube 120 to provide suction through the suction tube 120. This provides suction at the porous medium 140 for treatment of the defect.

In a second exemplary method, an endoscope is used to facilitate delivery into the subject.

This may comprise inserting the distal portion of the device 100 into the subject's nose and out through the mouth (as in the first exemplary method), prior to insertion into the endoscope. The device 100 may be inserted into the endoscope with the constraining sleeve 160 in the insertion position and the porous medium 140 in its compressed state. The endoscope is then inserted through the patient's mouth and towards the defect. Once the endoscope is located at, or proximal to, the defect, the sleeve 160 may be removed from the porous medium 140. For example, endoscopic graspers may be used to pull back/tear away the sleeve 160 from the porous medium 140. With the sleeve 160 removed from the porous medium 140, the endoscope may then be removed to enable the expanded porous medium 140 to be used to treat the defect (as described above for the first exemplary method).

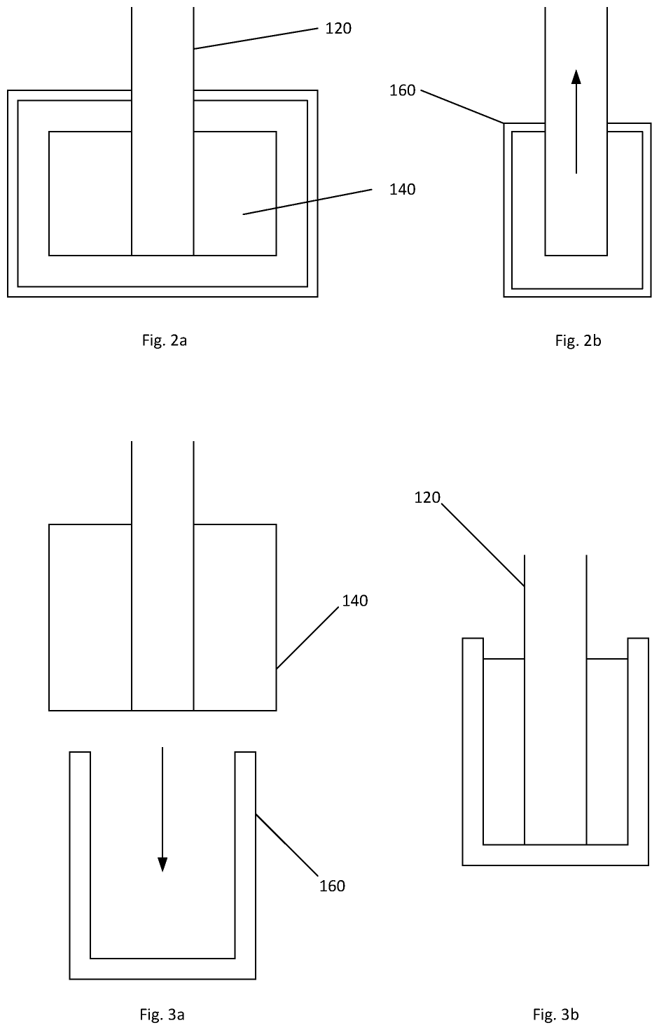

As described above, examples of the present disclosure may provide vacuum therapy devices for treatment of defects internal of a body, as well as providing vacuum therapy methods. Embodiments of the present disclosure are also directed to kits of parts for vacuum therapy devices, as well as methods of preparing vacuum therapy devices for use. Embodiments of the present disclosure are also directed to apparatuses and methods for preparing vacuum therapy devices. Such apparatuses and methods will now be described with reference to FIGS. 2, 3 and 4.

FIGS. 2a and 2b illustrate an exemplary method of preparing a vacuum therapy device.

The device of FIGS. 2a and 2b may comprise the vacuum therapy device 100 described above with reference to FIGS. 1a and 1b.

The constraining sleeve 160 may be shrinkable in response to application of a vacuum thereto. For example, the sleeve 160 may be of sufficient flexibility to enable it to collapse upon itself when suction is applied to its interior. The sleeve 160 may be configured to exist in a relaxed state in which the sleeve 160 is loose (e.g. so that it does not retain the porous medium 140 in the compressed state). The sleeve 160 may be placed on to the porous medium 140 in its relaxed state, as shown in FIG. 2a. As can be seen, there is a gap between the sleeve 160 and the porous medium 140, with the porous medium 140 in an expanded state. The sleeve 160 is configured to be placed onto the porous medium 140 in the insertion position to provide an (at least partially) airtight seal to the volume within the sleeve 160.

The device 100 is arranged so that, in response to suction being provided to the porous medium 140 via the suction tube 120, the sleeve 160 will begin to collapse around the porous medium 140. For example, the device 100 is arranged to provide a seal which is airtight enough for the sleeve 160 to begin to collapse onto the porous medium 140 in response to suction being applied to the porous medium 140. The sleeve 160 is configured to retain the porous medium 140 under compression as the sleeve 160 collapses in response to suction being applied thereto. As more suction is applied to the porous medium 140 (and thus more fluid in the porous medium 140, and between the porous medium 140 and the sleeve 160, is drawn away), the sleeve 160 is configured to collapse further causing the porous medium 140 to compress. The device 100 is arranged so that the sleeve 160 will keep shrinking, and the porous medium 140 will keep compressing, until the porous medium 140 is in its compressed state. The device 100 is configured so that the sleeve 160 will retain the porous medium 140 in its compressed state. For example, the device 100 may be operated to maintain the application of suction to the porous medium 140. As another example, the device 100 may be arranged to be sufficiently airtight so that when the suction is no longer applied to the porous medium 140, no fluid may enter the interior of the sleeve 160 to expand it. As another example, the material of the sleeve 160 may be selected so that, when shrunk, it remains in this state.

FIG. 2b shows the device 100 with suction being applied through the suction tube 120 (the arrow of FIG. 2b indicates fluid flow through the suction tube 120 away from the interior of the sleeve 160). The device 100 is configured to remain in this compressed state until the sleeve 160 is removed. For example, removal of the sleeve 160 may comprise forcefully having to remove the sleeve 160, such as by tearing it away, providing fluid into the interior of the sleeve 160 (e.g. through the suction tube 120), or pulling it off the porous medium 140. In this example, the arrangement of the suction tube 120, constraining sleeve 160 and porous medium 140 may provide a compression element configured to provide compression of the porous medium 140 (and to retain the porous medium 140 in its compressed state). The device 100, with the porous medium 140 retained in its compressed state, is then ready for insertion into a subject for treatment of the defect.

FIGS. 3a and 3b illustrate another exemplary method of preparing a vacuum therapy device.

The device of FIGS. 3a and 3b may comprise the vacuum therapy device 100 described above with reference to FIGS. 1a and 1b.

The constraining sleeve 160 of the device 100 shown in FIGS. 3a and 3b may be relatively rigid, and/or resistant to radial expansion (e.g. its cross-sectional area may not change). The sleeve 160 may be of sufficient rigidity so that forcing the porous medium 140 towards, and into, the sleeve 160 compresses the porous medium 140. For example, an end of the sleeve 160 into which the porous medium 140 is inserted for compression may be tapered (e.g. from wider to narrower) to facilitate compression. The sleeve 160 may be configured to resist lateral (e.g. radial) expansion while permitting bending of the sleeve 160. For example, the sleeve 160 may be manoeuvrable through the subject to defects in their GI tract by bending along its length, but at each point along its length the sleeve 160 is resistive to lateral expansion. The sleeve 160 may be configured to retain the porous medium 140 compressed, laterally, for insertion into the body. FIG. 3a shows the sleeve 160 with the porous medium 140 and distal end of the suction tube 120 being advanced thereto, and FIG. 3b shows the sleeve 160 retaining the porous medium 140 therewithin in its compressed state. The device 100, with the porous medium 140 retained in its compressed state, is then ready for insertion into a subject for treatment of the defect.

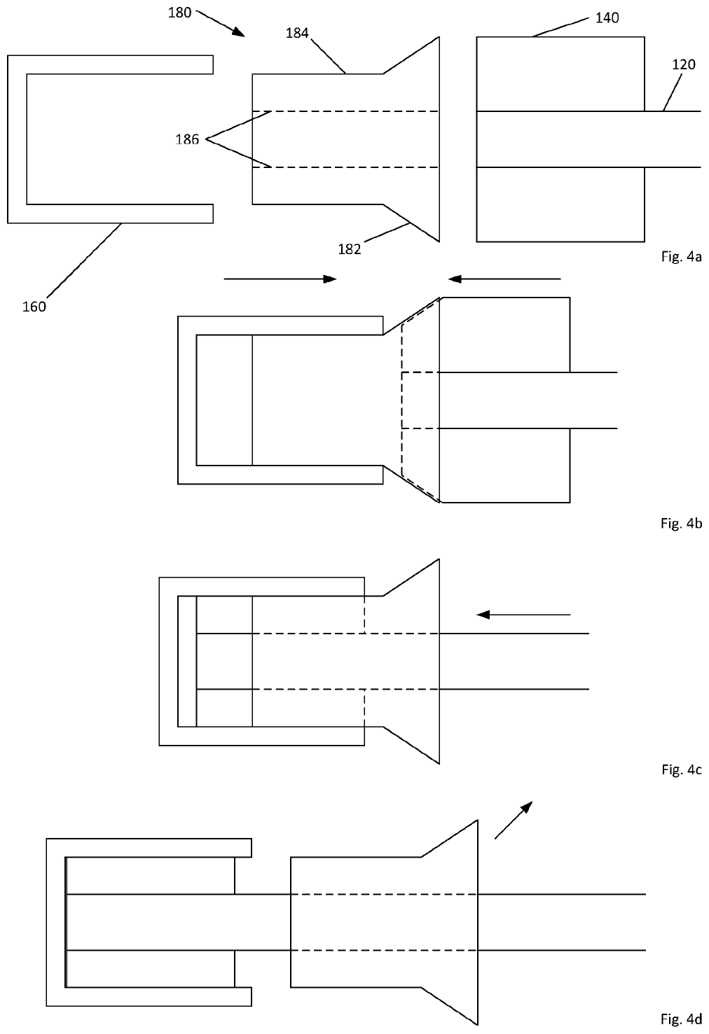

FIGS. 4a to 4d illustrate another exemplary method of preparing a vacuum therapy device.

The device of FIGS. 4a to 4d may comprise the vacuum therapy device 100 described above with reference to FIGS. 1a and 1b.

Additionally, a compression element is provided for facilitating compression of the porous medium 140 into its compressed state within the sleeve 160. The compression element comprises a funnel 180. The funnel 180 has a tapered region 182 and a sleeve connecting region 184, and also includes a removal channel 186.

The tapered region 182 is arranged at a porous medium 140 facing end of the funnel 180, and the sleeve connecting region 184 is arranged at a sleeve facing end of the funnel 180. The tapered region 182 comprises an inlet having an inlet cross-sectional area (an internal cross-sectional area for receiving a porous medium 140). The funnel 180 tapers down in cross-sectional area to a second cross-sectional area, which is less than the inlet cross-sectional area. The taper may be uniform, or it may vary in tapering rate. The sleeve connecting region 184 may be of a uniform cross-section along its length. For example, the sleeve connecting region 184 may have an internal cross-sectional area which is constant along its length, e.g. so that an outlet cross-sectional area for the funnel 180 corresponds to (e.g. is the same as) the second cross-sectional area. The removal channel 186 extends along a length of the funnel 180. The removal channel 186 comprises a gap in the surface/circumference of the funnel 180. The channel may extend radially outward in one direction from a central region of the funnel 180 (e.g. so that the funnel 180 is u-shaped when viewed in cross-section).

The funnel 180 is configured to facilitate compression of the porous medium 140. The funnel 180 is arranged so that a porous medium 140 forced through the inlet of the funnel 180 (into the tapered region 182) and towards the outlet (out from the sleeve connection region) will be compressed. The funnel 180 is arranged to provide sufficient compression of the porous medium 140 for insertion of the porous medium 140 into the sleeve 160.

The tapered section is configured to provide gradual compression of the porous medium 140 from a first, expanded, cross-sectional area, to a second, compressed, cross-sectional area. For example, the tapered section may taper from a cross-sectional area corresponding to the porous medium 140 when fully expand to a cross-sectional area corresponding to the porous medium 140 when sufficiently compressed for insertion into the sleeve 160. The tapered section is arranged so that a porous medium 140 received in the tapered section through the inlet will be compressed as it moves through the tapered section towards the outlet of the funnel 180.

The sleeve connecting region 184 is configured to provide an internal cross-sectional area for insertion of the porous medium 140 into the sleeve 160. An outer surface of the sleeve connecting region 184 is arranged to receive a sleeve 160 into which the porous medium 140 is to be inserted. For example, the funnel 180 may be configured to be at least partially inserted into a sleeve 160, e.g. so that the sleeve connection portion of the funnel 180 at least partially lies within the sleeve 160. The sleeve 160 and funnel 180 may be arranged so that a porous medium 140 compressed through the funnel 180 and passing out through the outlet will be received in the sleeve 160.

The removal channel 186 is arranged to run along a length of the funnel 180 (from the inlet to the outlet). A width of the removal channel 186 is selected based on a width of the suction tube 120. The removal channel 186 is configured to enable the funnel 180 to be removably coupled to the suction tube 120. For example, the funnel 180 may be pushed on to the suction tube 120 with the suction tube 120 moving through the removal channel 186 (e.g. radially inwards to a central region of the funnel 180). For example, the funnel 180 may then be pulled off the suction tube 120 with the suction tube 120 moving through the removal channel 186 (e.g. radially outwards from a central region of the funnel 180). The funnel 180 may be resilient, e.g. to enable the funnel 180 and/or channel to partially deform or bend for coupling to/removing from the suction tube 120. For example, the funnel 180 and removal channel 186 may be arranged to ‘snap on’ to a suction tube 120 (e.g. so that when the suction tube 120 is in a central region of the funnel 180, the edges of the channel are biased towards each other to provide a tighter seal for the suction tube 120 in the funnel 180). The removal channel 186 width may be selected to be slightly narrower than a width of the suction tube 120.

The device 100 may be arranged so that movement of the porous medium 140 and suction tube 120 through the funnel 180 urges the suction tube 120 into a central region of the funnel 180. The removal channel 186 may extend radially outward from the central region in one direction along the length of the funnel 180 (e.g. to provide a segment of the funnel 180 which has no material when viewed in cross-section). The device 100 and funnel 180 may be arranged so that once the porous medium 140 has been pushed through the funnel 180 and into the sleeve 160, the funnel 180 is mounted onto the suction tube 120. The funnel 180 will have the suction tube 120 running through its central region from inlet to outlet. The funnel 180 may be at least partially resilient and/or resistive, e.g. so that it remains gripped to the suction tube 120. The funnel 180 is configured to be pulled off a suction tube 120 radially outwardly. For example, the funnel 180 is configured to be pulled so that the suction tube 120 passes from the central region of the funnel 180 radially outwardly through the removal channel 186, thereby to remove the funnel 180 from the suction tube 120.

FIGS. 4a to 4d illustrate a method of using the funnel 180 to provide the porous medium 140 in the sleeve 160 in a compressed state.

FIG. 4a shows the three components (sleeve 160, funnel 180, porous medium 140 on suction tube 120) ready for coupling. As shown, the porous medium 140 is arranged adjacent to the inlet (e.g. porous medium 140 facing end of the funnel 180). The sleeve 160 is arranged adjacent to the outlet (e.g. sleeve facing end of the funnel 180).

FIG. 4b shows the porous medium 140 after having been advanced towards the funnel 180, and the sleeve 160 after having been advanced towards the funnel 180. It will be appreciated in the context of the present disclosure that these two acts need not be performed at the same time. The sleeve 160 is arranged to surround a portion of the sleeve connecting region 184, with the sleeve connecting region 184 inserted within the sleeve 160. A distal region of the porous medium 140 has begun to compress, as it is forced through the tapered section of the funnel 180.

FIG. 4c shows the sleeve 160 in place on the sleeve connecting region 184 and the porous medium 140 having been inserted all the way through the tapered region 182. The porous medium 140 is now at a small cross-sectional area for insertion into the sleeve 160. Some of the porous medium 140 has started protruding out through the outlet of the funnel 180 and into the interior of the sleeve 160, while some of the porous medium 140 remains in the funnel 180. The funnel 180 is located around the suction tube 120 with the suction tube 120 running along the central region of the funnel 180 (e.g. along its core/longitudinal axis). The removal channel 186 of the funnel 180 is also aligned with the central region of the funnel 180 (and thus the suction tube 120).

FIG. 4d shows the porous medium 140 in the sleeve 160 in its compressed state. The porous medium 140 has been inserted through the funnel 180 and into the sleeve 160.

Movement of the porous medium 140 and suction tube 120 through the funnel 180 and into the sleeve 160 has urged the sleeve 160 away from the funnel 180. The funnel 180 is then arranged on the suction tube 120 with the tube 120 running along the funnel's central (longitudinal) axis. The sleeve 160 and porous medium 140 are located at a distal end of the suction tube 120, with the funnel 180 coupled to the suction tube 120 in a more proximal region. The funnel 180 is then removed from the suction tube 120 by pulling the funnel 180 away (e.g. radially outward from the suction tube 120). The suction tube 120 will then move from the core of the funnel 180 radially outward through the removal channel 186. The funnel 180 may therefore be removed from the suction tube 120 without requiring it to be pulled off a proximal or distal end of the suction tube 120. The device 100, with the porous medium 140 retained in its compressed state, is then ready for insertion into a subject for treatment of the defect.

Vacuum therapy devices of the present disclosure may be configured for insertion into a patient to be delivered to, or proximal to, a defect internal of the patient's body. In examples, the vacuum therapy device 100 may be an endoscopic vacuum therapy device.

The device 100 may be arranged (e.g. sized and shaped) to be carried into the patient's body using an endoscope. For example, the device 100 may be arranged to be insertable into an endoscope, and/or the device 100 may be arranged so that it may be affixed to an endoscope (e.g. via suture so that the two are adjacent). The device 100 may be configured so that when inserted into the patient with an endoscope, the endoscope and device 100 (e.g. the endoscope housing the device 100, or the endoscope adjacent the device 100) may pass through bends in the internal lumens of the patient. For example, the device 100 may be configured for treatment of defects in a patient's gastrointestinal tract. For this, the device 100 may be configured to be inserted into a patient's nose (or their mouth, such as when the patient is already being ventilated). The device 100 may then be passed out of the patient's mouth where it may be inserted into an endoscope. The device 100 may be sufficiently flexible to pass round the bends on the way from the patient's nose or mouth into the defect their gastrointestinal tract. The device 100 may be arranged to be resistant to kinking during insertion into the patient's gastrointestinal tract.

The device 100 may be arranged to facilitate movement of the device 100 within a said endoscope (e.g. an outer surface of the device 100 may be configured to reduce friction with an endoscope, such as by having a friction-reducing coating). The device 100 may be arranged so that the endoscope and device 100 are separable when inside the patient (e.g. in response to force being applied to one of the endoscope or device 100, such as to tear a suture coupling the two, e.g. opening endoscopic graspers to release the suture). For example, the device 100 may be configured so that it is capable of twisting or bending with a radius of curvature of approximately 10 mm, such as 20 mm or less. For example, the device 100 may be configured to bend round 90 degrees or more without rupturing or kinking.

The device 100 may be configured to be inserted into the body via an additional outer housing or tube. An insertion applicator may be provided to provide passage into the subject's body. An insertion applicator may comprise an endoscope or other suitable tubing for controlling delivery of the device 100 into the subject's body. An insertion applicator may comprise tubing which may be pre-placed into a subject's body to facilitate insertion of the device 100 into the subject's body. For example, the insertion applicator may comprise a tube running through a subject's nasal passageway (e.g. in through their nostril) and towards their throat. The device 100 may be configured for insertion into such an insertion applicator. For example, the device 100 may have an outer coating on the suction tube 120 and/or sleeve 160 to facilitate movement of the suction tube 120, porous medium 140 and sleeve 160 through the applicator (e.g. the coating may comprise a friction-reducing coating).

The insertion applicator may comprise a friction-reducing coating on its inner surface (e.g. to permit movement of the suction tube 120, sleeve 160 and thus the porous medium 140, relative to the applicator). The inner lining may be made of a material arranged to reduce friction, such as fluorinated ethylene propylene. The inner lining may be adhered, or sewed, to the inside of the applicator, and/or it may just sit within the applicator without being affixed thereto.

In examples described herein, a constraining sleeve 160 may retain the porous medium 140 in a compressed state on the suction tube 120. However, it is to be appreciated in the context of the present disclosure that the particular form/arrangement of the constraining sleeves described herein need not be considered limiting. In some examples, a nasal insertion applicator may be provided for insertion into a subject's nasal passageway. The nasal insertion applicator may have an internal lumen through which the porous medium 140 and suction tube 120 may pass for insertion into the subject's body. For example, the distal portion of the suction tube 120 and the porous medium 140 may be inserted into the inner lumen of the nasal insertion applicator. The porous medium 140 and suction tube 120 may be advanced distally through the inner lumen of the applicator. They may then be passed further down the subject's GI tract, or back out the subject's mouth (e.g. for insertion into an endoscope). In some examples, the porous medium 140 may be retained in a compressed state by a constraining sleeve 160, which is separate to the nasal applicator. This sleeve 160 may then be removed as described herein (e.g. out the patient's mouth, when inside the patient etc.). In other examples however, the separate sleeve may not be provided, and instead the inner lumen of the nasal insertion applicator may provide a constraining sleeve which enables the porous medium 140 to be retained in its compressed state during insertion into the patient's nasal passageway. In which case, the inner lumen may be arranged to be inserted through a nasal passageway, and with an inner lumen sized and shaped to enable the porous medium 140 and suction tube 120 to be advanced therethrough. The nasal insertion applicator (and its inner lumen) may be configured to retain the porous medium 140 in its compressed state while the porous medium is advanced therethrough (e.g. the applicator may be sufficiently rigid to radial expansion to retain the porous medium 140 in its compressed state). Once the porous medium 140 has passed out the other end of the inner lumen (e.g. towards/in the patient's throat region, or further into the GI tract), the porous medium 140 may then expand. The inner lumen may have an anti-friction coating, as described herein.

The suction tube 120 may include a plurality of fenestrations (e.g. holes which provide a fluid flow path from the porous medium 140 into the hollow core of the suction tube 120). The fenestrations may be arranged along the length of the suction tube 120 which is inserted into the porous medium 140. The fenestrations may be distributed uniformly along the length of the suction tube 120, e.g. so that the number of fenestrations per unit length remains constant along the length of the suction tube 120 within the porous medium 140. The fenestrations may be distributed both radially and axially along the suction tube 120. In examples, the suction tube 120 may have a tapered structure in the region where it is inserted into the porous medium 140 (where it tapers from a wider diameter to a narrower diameter). In this region, the fenestrations may only be in the narrowed portion of the suction tube 120 (on the side of the tapering with the smaller diameter). In other examples, there may be no tapering of the suction tube 120, and/or the fenestrations may be located on any part of the suction tube 120, e.g. they may only be located in or proximal to the region of the suction tube 120 which is inserted into the porous medium 140.

The arrangement of fenestrations along the suction tube 120 may be selected so that the same, or a similar, amount of suction is provided at locations along the length of the suction tube 120. The cross-sectional area of fenestrations may be selected based on the cross-sectional area of the suction tube 120, such as to inhibit suction being predominantly delivered to fenestrations closer to the source of negative pressure. For example, the cross-sectional area of fenestrations may increase the further down the suction tube 120 the fenestrations are. The number or density of fenestrations may also vary along the length of the suction tube 120. For example, the suction tube 120 may be arranged so that the cross-sectional area of fenestrations per unit length of suction tube 120 increases from its proximal end to its distal end, so that fenestrations at the proximal end do not use a disproportionately high amount of the suction (so suction is still provided at the distal end).

The suction tube 120 may extend from a connection to a source of negative pressure to the porous medium 140. For example, the suction tube 120 may comprise a single tube which couples the source of negative pressure to the porous medium 140. In other examples, additional components may be provided along the fluid flow path which couples the negative pressure source to the porous medium 140. In examples, where the suction tube 120 directly couples the porous medium 140 to the source of negative pressure, the suction tube 120 may have fenestrations therein, and these may only be located at a distal end of the tube 120, e.g. where the porous medium 140 will be coupled to the tube 120.

Constraining sleeves of the present disclosure may be arranged to facilitate their movement from the insertion position in which they retain the porous medium 140 in its compressed state to a treatment position for expansion of the porous medium 140. The sleeve 160 may comprise a gripping region and/or gripping element configured to be gripped for movement of the constraining sleeve 160. For example, the gripping element may comprise one or more tabs, such as tabs which extend at least partially radially outward from the sleeve 160 to be grasped to facilitate movement of the sleeve 160 to the treatment position. The gripping region may comprise a region which is grippier, and/or provides an easier region for a user to grasp to move the sleeve 160. Sleeves of the present disclosure may be relatively small (e.g. substantially smaller than the length of the suction tube 120). Sleeves may cover only a small portion of the suction tube 120. For example, sleeves of the present disclosure may be unlike catheters (e.g. sleeves of the present disclosure may not comprise catheters), or other similar components which extend the length, or at least a majority of the length, of the suction tube 120. For devices of the present disclosure, the outer surface of the suction tube 120 and/or sleeve 160 may form an outer surface of the device 100 for insertion into the body (either as the device 100 by itself, or inside an endoscope).

Sleeves may be arranged to be torn away from the porous medium 140. For example, the sleeves may be arranged so that they can be ripped away from the porous medium 140. For this, a pre-defined line or region of weakness may be provided in the sleeve 160, such as a perforated region to facilitate easier ripping. The sleeve 160 may be configured to retain the porous medium 140 in a radially compressed while enable the sleeve 160 to be torn away by user interaction to enable expansion of the porous medium 140.

The device 100 may comprise one or more graspable elements such as sutures. The graspable elements may be arranged to facilitate movement of the porous medium 140. For example, the sutures may be graspable by grippers of an endoscope to facilitate controlling movement of the porous medium 140 within the body. The graspable elements may also facilitate movement of the sleeve 160 (e.g. away from its insertion position).

In examples described herein, devices of the present disclosure may be inserted into the body through the nasal passageway. However, it is to be appreciated that this is not limiting, as devices of the present disclosure may still be inserted through the mouth. For example, in some circumstances, more than one device may be inserted into a patient at a time. In the event that a plurality of devices are to be inserted, this insertion may go through the patient's mouth, rather than through their nose.

In examples shown in the FIGS., the sleeves of the present disclosure are shown with proximal and distal ends which cover proximal and distal surfaces (respectively) of the porous medium 140. However, it is to be appreciated in the context of the present disclosure that one or both of the proximal and distal covering ends of the sleeve 160 need not be provided. For example, the sleeve 160 may be annular with one or no ends. The porous medium 140 may be configured to slide into the sleeve 160 without abutting the sleeve 160 at one or both ends. In other examples, the sleeve 160 may have only one end present. Sleeves of the present disclosure may have a closed, or at least partially closed, distal end (e.g. so that the porous medium 140 may push the sleeve 160 away from a funnel 180 when inserted into the sleeve 160 using the funnel 180). Sleeves may have an open proximal end, e.g. to enable a porous medium 140 to be inserted into the sleeve 160 through an opening at the proximal end.

In examples described herein, the porous medium 140 (and sleeve 160, if connected) are located at a distal portion of the suction tube 120. The distal portion of the suction tube 120 comprises a portion of the suction tube 120 which will be inserted into the subject and located proximal to the defect internal of their body. In the examples shown in the FIGS., the distal portion of the suction tube 120 comprises a distal end (a distal tip) of the suction tube 120, e.g. so that no tubing protrudes further distally from that distal end. However, it is to be appreciated that this not be considered limiting, as the tube 120 may extend beyond the distal end of the porous medium 140. For example, the suction tube 120 may extend further into the subject's body to facilitate removal of fluids further into the body (e.g. further into the GI tract) than the location at which the porous medium 140 is placed. In such examples, one or more fenestrations may be provided in the suction tube 120 at the region of the suction tube 120 which is adjacent to (e.g. runs through a hollow passageway of) the porous medium 140. Alternatively, or additionally, the tube 120 may split at that region to provide an additional length of tubing extending distally beyond the distal end of the porous medium 140 and to provide tubing for providing suction at the porous medium 140.

In examples described herein, the distal tip of the suction tube 120 is aligned with the distal end of the porous medium 140. However, this is not to be considered limiting. For example, as described above, the suction tube 120 may extend distally beyond the distal end of the porous medium 140. In other examples, the distal end of the suction tube 120 may not extend as far as the distal end of the porous medium 140. For example, there may be a region of porous medium 140 at the distal end of the device 100 to provide a softer leading edge for insertion into the body. The suction tube 120 may extend a portion of the way along the length of the porous medium 140 (e.g. through its hollow passageway), e.g. to provide a distal region of the porous medium 140 without a suction tube 120 therein, but to provide suction to a majority of the volume of the porous medium 140.

Examples of the present disclosure are configured for treatment of a defect internal of a human or animal body. For example, embodiments may be configured for treatment of an adult human. Embodiments may be configured for insertion into an adult human's GI tract via their nasal passageway. For example, devices may be configured to fit within a patient's nostril and through their nasal passageway towards their throat. Devices of the present disclosure may be sized and shaped to fit within an adult human nasal passageway (and to be able to move through their nasal passageway). Devices of the present disclosure may be sufficiently flexible to bend round the curves in the body (e.g. when travelling from the nostril to the throat via the nasal passageway).

Embodiments of the present disclosure are directed to vacuum therapy devices and methods for treatment of a defect internal of a human or animal body. In particular, embodiments include a porous medium which may be deployed from an insertion position to a treatment position for providing treatment of the defect. When the porous medium is deployed into its treatment position, the porous medium will be in an expanded state for providing treatment of the defect. As disclosed herein, treatment of the defect is provided by applying suction to the porous medium. In turn, this may cause one or more surfaces in the internal lumen of the patient to come into contact with the porous medium. This approach may facilitate healing of any surfaces which come into contact with the porous medium. As such, embodiments may find utility for healing a number of different types of defect internal of a human or animal body. Embodiments may provide responsive treatment (e.g. treating an area in response to there being an issue with that area which needs fixing) and/or preventative treatment (e.g. treating a weaker area which may be more susceptible to subsequent damage despite not currently being actively in need of treatment per se.).

One example of a type of defect for which embodiments may provide treatment is a cavity in a body lumen of the patient. For example, the cavity may be in a patient's nasogastric lumen (e.g. somewhere between their nose and stomach. Another example of a type of defect for which embodiments may provide treatment is neighbouring tissue which is close to another defect (e.g. tissue which is proximal to and/or surrounds a defect such as a cavity). Another example of a type of defect for which embodiments may provide treatment is a pre-defined area of weakness or vulnerability within the body. In other words, treatment may be applied to a portion of an internal lumen of a human or animal body which may have a region which is more susceptible to damage. For example, such a region may comprise a surgical join, where two portions of the lumen have been stitched together. Treatment may be applied to such an area to help facilitate healing and strengthening of that area. That is, the porous medium may be inserted where there is not currently an active issue which needs treating (such as a cavity), but instead the porous medium may be inserted to try to inhibit any active issue from developing in that region. In relation to use of devices for preventative reasons, one example use case for this is for treating an anastomosis, such as a surgical join made in the GI tract (e.g. after an oesophagectomy). Embodiments may be used to inhibit the likelihood of that surgical join rupturing by bringing the surgical join into contact with the porous medium, and applying suction thereto.

It will be appreciated in the context of the present disclosure that the particulars for deployment of the porous medium may vary depending on the defect to be treated. Relative movement between the porous medium and the constraining sleeve may enable the porous medium to expand from an insertion position to a treatment position. This deployment may be controlled to occur at or proximal to the defect to be treated. Applying suction to the porous medium may draw in surrounding body tissue towards the porous medium, and so the porous medium may be deployed in a location where the application of suction to the porous medium will bring the region to be treated closer to, or into contact with, the porous medium. The porous medium may be deployed directly into the region to be treated, e.g. it may be inserted directly into a defect such as a cavity. Application of suction to the porous medium may then provide some collapsing of the cavity towards the porous medium.

However, the porous medium need not be inserted directly into a cavity—the application of suction to a porous medium located proximal to the cavity may draw the cavity towards the porous medium as desired. In other examples, the area to be treated by the porous medium need not include a cavity. For example, the area to be treated may be tissue towards the edge of the defect, such as tissue surrounding a cavity. In which case, the porous medium may be deployed so that it is in contact with that tissue, or it may be deployed to a location proximal to that tissue (e.g. so that application of suction to the porous medium brings the tissue into contact with the porous medium). In other words, the porous medium may be deployed inside the body in a position which is aligned with the area to be treated (e.g. so that suction applied to the porous medium brings the area to be treated into contact with the defect). Similarly, where there is an area of weakness to be treated (e.g. a surgical join), the porous medium may be deployed and positioned to be in contact with that area of weakness or to be located proximal to that area of weakness (e.g. so that the presence of the porous medium, and the application of suction thereto, may act to inhibit any further damage occurring to that region).

It is to be appreciated in the context of the present disclosure that the particular order of events relating to removal of the sleeve 160 and insertion of the distal end of the device 100 into the body need not be considered limiting. Sleeves of the present disclosure may enable a porous medium 140 to be retained in a compressed state. For example, this compressed state may be sufficiently compressed for insertion of the porous medium 140, sleeve 160 and distal end of the suction tube 120 through a subject's nose. Sleeves of the present disclosure may thus facilitate insertion of the distal end of the device 100 (with porous medium 140) into and through a subject's nasal passageway. The sleeve 160 may then be removed prior to using the expanded porous medium 140 for treatment of the defect. When and where the sleeve 160 is removed may vary. For example, as described above, the distal end of the suction tube 120 with sleeve 160 and porous medium 140 may be passed back out through a patient's mouth where the sleeve 160 may be removed. As another example, the sleeve 160 may be removed inside a patient, e.g. using surgical components such as a grasper of an endoscope. The porous medium 140 may be passed into the subject's body towards the defect with the sleeve 160 on, or not, and/or the porous medium 140 may be passed into an endoscope for insertion into a subject's body with the sleeve 160 on, or not.

It is to be appreciated that the particular funnel arrangement described above need not be considered limiting. For example, the particular tapering of the funnel 180 may vary, e.g. there may be a consistent tape, and/or there may be a varied taper. Likewise, the funnel 180 may have a sleeve receiving portion, or it may not. For example, a physician could simply hold the sleeve 160 next to a distal (sleeve-facing) end of the device 100, such that a porous medium 140 is passed through the inlet, compressed, and then moves through the outlet into the sleeve 160. Funnels of the present disclosure may be configured to facilitate compression of the porous medium 140 for insertion into a sleeve 160. They may also be configured to be removable from a suction tube 120 without needing to slide off a distal or proximal end of the suction tube 120.

It is to be appreciated in the context of the present disclosure that, to the extent that certain methods may be applied to the living human or animal body, it will be appreciated that such methods may not provide any surgical or therapeutic effect. In addition, it will be appreciated that such methods may be applied ex vivo, to tissue samples that are not part of the living human or animal body. For example, the methods described herein may be practiced on meat, tissue samples, cadavers, and other non-living objects.

It will be appreciated from the discussion above that the examples shown in the figures are merely exemplary, and include features which may be generalised, removed or replaced as described herein and as set out in the claims. As will be appreciated by the skilled reader in the context of the present disclosure, each of the examples described herein may be implemented in a variety of different ways. Any feature of any aspects of the disclosure may be combined with any of the other aspects of the disclosure. For example method aspects may be combined with apparatus aspects, and features described with reference to the operation of particular elements of apparatus may be provided in methods which do not use those particular types of apparatus. In addition, each of the features of each of the examples is intended to be separable from the features which it is described in combination with, unless it is expressly stated that some other feature is essential to its operation. Each of these separable features may of course be combined with any of the other features of the examples in which it is described, or with any of the other features or combination of features of any of the other examples described herein. Furthermore, equivalents and modifications not described above may also be employed without departing from the invention.

Other examples and variations of the disclosure will be apparent to the skilled addressee in the context of the present disclosure.

1. A vacuum therapy device for treatment of a defect internal of a human or animal body, the device comprising:
a suction tube extending from: (i) a proximal portion for coupling to a source of negative pressure outside the body, to (ii) a distal portion for providing suction proximal to the defect when inserted into the body;
a porous medium coupled to the distal portion of the suction tube to provide suction to the defect when inserted into the body for treatment thereof; and
a constraining sleeve for selective compression of the porous medium, the constraining sleeve having a proximal end and a distal end, wherein the constraining sleeve is movable relative to the porous medium between:
an insertion position in which the proximal and distal ends of the sleeve are arranged at the distal portion of the suction tube so that the sleeve at least partially surrounds the porous medium to retain the porous medium in a compressed state during insertion into the body; and
a treatment position in which the porous medium is in an expanded state for treatment of the defect.

2. The vacuum therapy device of claim 1, wherein the constraining sleeve is configured to be inserted through a nasal passageway of a human.

3. The vacuum therapy device of claim 2, wherein the constraining sleeve is configured to retain the porous medium in its compressed state during insertion of said porous medium and the distal portion of the suction tube through the nasal passageway.

4. The vacuum therapy device of claim 3, wherein the device is arranged to enable expansion of the porous medium to its expanded state once the porous medium and distal portion of the suction tube have passed through the nasal passageway.

5. The vacuum therapy device of claim 4, wherein the device is configured to enable the distal portion of the suction tube, the porous medium and the constraining sleeve to be passed out through the human mouth after being inserted through their nasal passageway for movement of the constraining sleeve from the insertion position to the treatment position.

6. The vacuum therapy device of claim 1, wherein the constraining sleeve comprises a gripping region and/or a gripping element configured to be gripped for movement of the constraining sleeve from the insertion position to the treatment position.

7. The vacuum therapy device of claim 6, wherein the gripping element of the constraining sleeve comprises a tab.

8. The vacuum therapy device of claim 7, wherein the tab extends radially outward from the constraining sleeve.

9. The vacuum therapy device of claim 1, wherein the porous medium, the constraining sleeve and the distal portion of the suction tube are configured to be inserted into a tube of a nasal insertion applicator and/or endoscope for placement into the body.

10. (canceled)

11. The vacuum therapy device of claim 1, wherein the constraining sleeve comprises an inner lining configured to reduce friction between the sleeve and the porous medium.

12. The vacuum therapy device of claim 1, wherein the constraining sleeve is small relative to the length of the suction tube.

13. The vacuum therapy device of claim 1, wherein the constraining sleeve is configured to move distally relative to the suction tube from the insertion position to the treatment position or to be torn away from the porous medium.

14. The vacuum therapy device of claim 1, wherein the device is arranged so that movement of the constraining sleeve from the insertion position to the treatment position causes the porous medium to expand into its expanded state for treatment of the defect.

15. The vacuum therapy device of claim 1, wherein the suction tube comprises a vacuum coupling for connection to a pump to provide suction to the porous medium.

16. The vacuum therapy device of claim 1, wherein the constraining sleeve is configured to shrink in response to suction being provided within the sleeve.

17. The vacuum therapy device of claim 16, wherein the constraining sleeve is configured to shrink and compress the porous medium into its compressed state when the sleeve surrounds the porous medium and suction is applied to the porous medium by the suction tube.

18-21. (canceled)

22. A kit of parts for nasal insertion of a vacuum therapy device into a human or animal body for treatment of a defect in the body, the kit comprising:
a suction tube extending from: (i) a proximal portion for coupling to a source of negative pressure outside the body, to (ii) a distal portion for providing suction proximal to the defect when inserted into the body;
a porous medium couplable to the distal portion of the suction tube to provide suction to the defect when the device is assembled and inserted into the body for treatment thereof; and
a nasal insertion applicator having an internal lumen which provides a constraining sleeve for selective compression of the porous medium, wherein the nasal insertion applicator is configured to be placeable into an insertion position within a nasal passageway of the body to enable the distal portion of the suction tube and the porous medium to pass through the applicator into the body while retaining the porous medium in a compressed state during insertion into the body.

23. A method of preparing a vacuum therapy device for treatment of a defect internal of a human or animal body, the device comprising: (i) a suction tube extending from a proximal portion for coupling to a source of negative pressure outside the body to a distal portion for providing suction proximal to the defect when inserted into the body, and (ii) a porous medium coupled to the distal portion of the suction tube to provide suction to the defect when inserted into the body for treatment thereof, the method comprising:
providing the porous medium in a compressed state within a constraining sleeve with the sleeve in an insertion position in which proximal and distal ends of the sleeve are arranged at the distal portion of the suction tube so that the sleeve at least partially surrounds the porous medium to enable the sleeve to retain the porous medium in the compressed state during insertion into the body, wherein the constraining sleeve is movable relative to the porous medium between the insertion position and a treatment position in which the porous medium is in an expanded state for treatment of the defect.

24. The method of claim 23, wherein providing the porous medium in the compressed state within the constraining sleeve comprises placing the constraining sleeve over the porous medium and applying suction to the porous medium through the suction tube to shrink the sleeve.

25. The method of claim 23, wherein providing the porous medium in the compressed state within the constraining sleeve comprises moving the porous medium through a funnel from a larger cross-sectional area to a smaller cross-sectional area, wherein at the smaller cross-sectional area the porous medium is in its compressed state and it fits within the constraining sleeve.
